(19) United States
(12) Patent Application Publication
(10) Pub. No.: US 2026/0364244 A1
(43) Pub. Date: Nov. 19, 2026

(54) Drainage Bag with Vacuum Metering Chamber

(71) Applicant: C. R. Bard, Inc., Franklin Lakes, NJ (US)

(72) Inventors: Varad Chavan, Kolhapur (IN); Rohit Sinha, Lawrenceville, GA (US); Jason Jishen Cheng, Avondale Estates, GA (US); David M. Simiele, Roswell, GA (US)

(21) Appl. No.: 19/145,822

(22) PCT Filed: Jan. 10, 2023
(86) PCT No.: PCT/US23/10515

§ 371 Date: Jul. 3, 2025

(51) Int. Cl. A61M 1/00; A61M 25/00

(52) U.S. Cl. CPC A61M 1/782; A61M 1/69; A61M 1/73; A61M 25/0017; A61M 2205/3327; A61M 2205/3379

(57) ABSTRACT

A drainage system for draining a liquid from a patient includes a drainage tube coupled between a catheter and a metering chamber, where the metering chamber includes a vacuum compartment and measuring compartment having a drain port disposed the between. A collection container is coupled with the measuring compartment via a transfer port that enables a clinician to pour drainage liquid from the measuring compartment into the collection container. An air pump is coupled with the vacuum compartment such that vacuum created by the air pump, when activated, (i) transitions the plunger valve from a normally open state to a closed state and (ii) induces flow of pooled drainage liquid along the drainage tube toward the vacuum chamber. The metering chamber provides for repeated collecting, measuring, and emptying of the drainage liquid. A sample port of the measuring compartment provides for sampling of the drainage liquid collected therein.

### BACKGROUND

The draining of liquid (e.g., urine) from a patient may include the use of a liquid drainage system including a flexible drainage tube extending from a drainage catheter to a collection container. Typical catheters include indwelling catheters, Foley catheters, balloon catheters, peritoneal drainage catheters, or the like, and are configured to be inserted into an orifice within the body of a patient to drain a liquid therefrom. In some instances, the flexibility of the drainage tube can form sections of positive incline, also termed “dependent loops,” along the drainage tube where drainage liquid can accumulate. Liquid pooling within dependent loops can cause various complications. For example, urine pooling can be a source of catheter associated urinary tract infection (“CAUTI”) causing agents such as bacteria, microbes, and the like. Hospital Acquired Infections (“HAI”), such as CAUTI, are detrimental to the patient, and also incur extra costs in treating these additional complications. Embodiments disclosed herein are directed to clearing drainage liquid from dependent loops thereby, reducing patient risk.

### SUMMARY

Briefly summarized, disclosed herein is a drainage system for draining a liquid from a patient. The system, according to some embodiments, includes a drainage tube configured to receive a drainage liquid from the patient, where a distal end of the drainage tube configured to couple with a catheter; and a metering chamber coupled with the drainage tube at a proximal end of the drainage tube. The metering chamber includes a vacuum compartment and a measuring compartment. The vacuum compartment includes (i) an inlet port coupled with the drainage tube, (ii) a vacuum port, and (iii) a drain port having a plunger valve in line therewith. The measuring compartment is fluidly coupled with the vacuum chamber via the drain port. The system further includes a collection container coupled with the metering chamber via a transfer port, where the transfer port is configured to enable selective transfer of the drainage liquid from the measuring compartment to the collection container. The system further includes an air pump assembly that includes (i) an air pump having an air inlet and an air outlet, (ii) an air inlet tube coupled between the air inlet and the vacuum port, and (iii) an air outlet tube coupled between the air outlet and an air port of the drainage tube adjacent the distal end. Activation of the air pump induces flow of the drainage liquid along the drainage tube toward the vacuum chamber. Upon deactivation of the air pump, the drainage liquid drains from the vacuum compartment into the measuring compartment through the plunger valve.

In some embodiments, the plunger valve is configured to transition between an open state when the air pump is deactivated and a closed state when the air pump is deactivated.

In some embodiments, the plunger valve comprises a plunger displaceable between a “down” position in accordance with the plunger valve disposed in the open state, and an “up” position in accordance with the plunger valve disposed in the closed state. In some embodiments, the plunger comprises a plurality of deflectable posts coupled with a circular base, where the posts extend vertically away from the circular base. In some embodiments, the posts are coupled with the drainage port via a snap fit coupling, the snap fit coupling configured to limit downward displacement of the plunger to define the “down” position.

In some embodiments, the circular base comprises an annular sealing ledge extending along a circumference of the circular base, where the annular sealing ledge is configured to form a seal with a corresponding annular sealing edge of the drain port when the plunger is disposed in the “up” position, and the seal defines the closed state of the plunger valve.

In some embodiments, activation of the air pump defines a vacuum within the vacuum compartment, where the vacuum exerts an upward force on the plunger to displace the plunger to the “up” position, and where deactivation of the air pump relieves the vacuum within the vacuum compartment, thereby allowing the plunger to self-displace to the “down” position.

In some embodiments, the metering chamber is attached to a front side of the collection container.

In some embodiments, a front wall of the measuring compartment includes volumetric graduation marks configured to indicate a volume of the drainage liquid contained within the measuring compartment, and in some embodiments, a front wall of the collection container includes volumetric graduation marks configured to indicate a volume of the drainage liquid contained within the collection container.

In some embodiments, the transfer port is located above a defined maximum liquid level of the measuring compartment, and in some embodiments, the transfer port is located above a defined maximum liquid level of the collection container.

In some embodiments, a bottom wall of the vacuum compartment is sloped toward the drain port to facilitate complete drainage of the drainage liquid from the vacuum compartment.

In some embodiments, a bottom wall of the measuring compartment includes a sample port, and the bottom wall of the of the measuring compartment is sloped toward the sample port to facilitate sampling of the drainage liquid within the measuring compartment.

In some embodiments, the system further includes an isolation valve disposed in line with the drainage tube at the distal end of the drainage tube, where the isolation valve is configured to prevent fluid flow toward the catheter. In some embodiments, the isolation valve is configured to transition between a closed state and open state in accordance with activation and deactivation of the air pump, respectively. In some embodiments, the isolation valve is a check valve configured to allow proximal fluid flow through the isolation valve and prevent distal fluid flow through the isolation valve.

In some embodiments, the system further includes a connector disposed at the distal end of the drainage tube, where the air port and the isolation valve are integral to the connector.

In some embodiments, the system further includes a vacuum-port sterilizing filter disposed in line with the vacuum port, where the vacuum-port sterilizing filter is configured to inhibit contamination of the metering chamber via the vacuum port. In some embodiments, the system further includes an air-port sterilizing filter disposed in line with the air port, where the air-port sterilizing filter is configured to inhibit contamination of the drainage tube via the air port.

Also disclosed herein is a method of draining liquid from a patient that, according to some embodiments, includes (i) providing a drainage system including a drainage tube extending between a catheter and a metering chamber; (ii) establishing a passive flow of a drainage liquid from the patient along the drainage tube; (iii) activating an air pump to transport pooled drainage liquid along the drainage tube to the metering chamber, where activating the air pump defines a pressure difference across the pooled drainage liquid within the drainage tube, and where activating the air pump also transitions a plunger valve disposed between a vacuum compartment and a measuring compartment of the metering chamber from an open state to closed state. The method further includes recording a volumetric measurement the drainage liquid contained within the metering chamber and pouring the drainage liquid from the metering chamber into a collection container of the drainage system through a transfer port disposed between the metering chamber and the collection container.

In some embodiments, the method further includes deactivating the air pump to transition the plunger valve from the closed state to the open state, where the plunger valve in the open state defines a fluid flow path between the vacuum compartment and the measuring compartment.

In some embodiments of the method, the metering chamber is located in front of the collection container during use. In some embodiments of the method, the metering chamber includes a rigid container, and the collection container includes a flexible bag.

In some embodiments, the method further includes collecting overflowing drainage liquid within the collection container, where the overflowing drainage liquid exits the measuring compartment via the transfer port.

In some embodiments of the method, the metering chamber is attached to a front wall of the collection container such that the metering chamber is suspended from the front wall during use.

Also disclosed herein is a urine collection bag assembly that, according to some embodiments, includes a metering chamber formed of a rigid structure coupled with a drainage tube, where the drainage tube is configured to couple with a urinary catheter. The assembly further includes a collection bag fluidly coupled with the metering chamber via a transfer port, where the transfer port is configured to enable selective transfer of urine from the metering chamber to the collection bag. In such embodiments, a back wall of the metering chamber is attached to a front wall of the collection bag, and the transfer port extends between the back wall and the front wall.

In some embodiments, the metering chamber includes a vacuum compartment that includes (i) an inlet port coupled with the drainage tube, (ii) a vacuum port configured to couple with an inlet air tube of an air pump, and (iii) a drain port having a plunger valve in line therewith. The metering chamber further includes a measuring compartment fluidly coupled with the vacuum chamber via the drain port.

In some embodiments of the assembly, the plunger valve is configured to transition (i) from a normally open state to a closed state when a vacuum is defined within the vacuum chamber and (ii) from the closed state to the open state when the vacuum is removed from the vacuum chamber.

In some embodiments of the assembly, a front wall of the metering chamber includes volumetric graduation marks configured to indicate a volume of the drainage liquid contained within the measuring compartment, and in some embodiments, a front wall of the collection container includes volumetric graduation marks configured to indicate a volume of the drainage liquid contained within the collection container.

In some embodiments of the assembly, the transfer port is located above a defined maximum liquid level of the measuring compartment.

In some embodiments of the assembly, a bottom wall of the measuring compartment includes a sample port, and the bottom wall of the of the measuring compartment is sloped toward the sample port to facilitate sampling of the drainage liquid within the measuring compartment.

In some embodiments, the assembly further includes an isolation valve disposed in line with the drainage tube, where the isolation valve configured to prevent fluid flow toward the catheter.

In some embodiments, the assembly further includes a connector disposed at the distal end of the drainage tube, where the connector includes an air port in fluid communication with the drainage tube, and where the air port is configured to couple with an air outlet tube of the air pump.

In some embodiments, the assembly further includes a vacuum-port sterilizing filter disposed in line with the vacuum port, where the vacuum port sterilizing filter is configured to inhibit contamination of the metering chamber via the vacuum port. In some embodiments the assembly further includes an air-port sterilizing filter disposed in line with the air port, where the air-port sterilizing filter is configured to inhibit contamination of the drainage tube via the air port.

These and other features of the concepts provided herein will become more apparent to those of skill in the art in view of the accompanying drawings and the following description, which describe particular embodiments of such concepts in greater detail.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a liquid drainage system for draining liquid from a patient, in accordance with some embodiments disclosed herein.

FIG. 2 is a front view of a metering chamber of the system of FIG. 1, in accordance with some embodiments disclosed herein.

FIGS. 3A-3C are various detailed views of a plunger valve of the metering chamber of FIG. 2, in accordance with some embodiments disclosed herein.

FIG. 4A is a cross-sectional side view illustration of the metering chamber and the collection container illustrating an instance of use that includes pouring drainage liquid from the metering chamber into the collection container, in accordance with some embodiments disclosed herein.

FIG. 4B is a cross-sectional side view illustration of the metering chamber and the collection container illustrating another instance of use that includes drainage liquid overflowing from the metering chamber to the collection container, in accordance with some embodiments disclosed herein.

FIG. 5 is a block diagram of a method of draining liquid from the patient utilizing the system of FIG. 1, in accordance with some embodiments disclosed herein.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to” and “coupled with” refer to any form of interaction between two or more entities, including mechanical, electrical, magnetic, electromagnetic, fluid, and thermal interaction. Two components may be connected or coupled with each other even though they are not in direct contact with each other. For example, two components may be coupled with each other through an intermediate component.

The directional terms “proximal” and “distal” are used herein to refer to opposite locations on a medical device. The proximal end of the device is defined as the end of the device closest to the end-user when the device is in use by the end-user. The distal end is the end opposite the proximal end, along the longitudinal direction of the device, or the end furthest from the end-user. The term “fluid” as used herein may refer to either a gas or a liquid.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

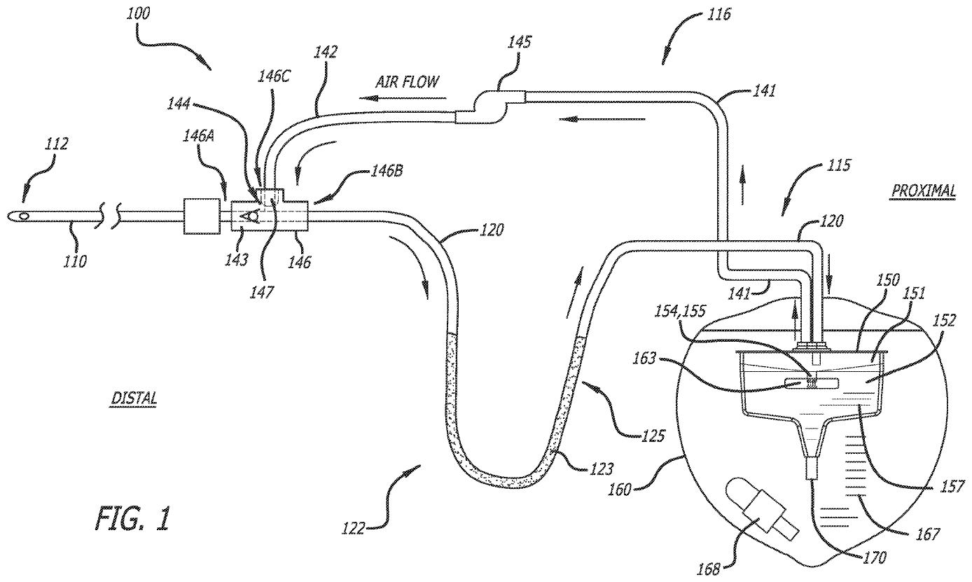

FIG. 1 shows an exemplary drainage system (“system”) 100, including a catheter 110, a drainage tube 120, a metering chamber 150, and a collection container 160. The catheter 110 includes an eyelet 112 that provides fluid communication with a lumen of the catheter 110, and is configured to drain a drainage liquid (liquid) 123 from a liquid source within a patient, e.g., a bladder. In general, the system provides a drainage pathway to transport liquid 123 from the catheter 110 to the collection container 160. The system 100 further provides for volumetric measurement (including repeated measurement) of the liquid 123 during and/or at the conclusion of a treatment period.

The drainage tube 120 extends from the catheter 110 to the metering chamber 150. A transfer port 163 provides for transferring the liquid 123 from the metering chamber 150 to the collection container 160. In use, the liquid 123 from the patient may flow from the catheter 110, through the drainage tube 120, and into the metering chamber 150. The liquid 123 may then be transferred from the metering chamber 150 to the collection container 160 through the transfer port 163. The drainage tube 120 can be formed of rubber, plastic, polymer, silicone, or similar suitable material. In the illustrated embodiment, the collection container 160 includes a flexible collection bag. However, in other embodiments, the collection container 160 may include a rigid container or similar suitable container for collecting a liquid, e.g., urine, drained from the patient via the catheter 110. In operation, the drainage system 100 may facilitate a passive draining process of the liquid 123 from the patient without incident. In some instances, one or more complications may arise during the passive draining process requiring corrective action as further described below.

As shown in FIG. 1, the flexibility of the drainage tube 120 can result in sections of the drainage tube 120 having one or more dependent loops 122 followed by a positive incline section 125. The positive incline section 125 can lead to liquid pooling (i.e., accumulation) within the dependent loop 122 of the drainage tube 120. A dependent loop 122 may be any portion of the drainage tube 120 that is lower than a downstream portion so as to create a positive incline 125 relative to the direction of fluid flow. Dependent loops 122 can form in slack portions of the drainage tube 120. The dependent loop 122 may be a complete loop, a partial loop, or any segment of the drainage tube 120 that causes the liquid 123 to pool in the drainage tube 120.

In instances of pooling, a drainage tube clearing process, as described herein, may provide a corrective action to the pooling of the liquid 123. In the illustrated embodiment, an air pump 145 can provide a pressure difference across a pooled volume of the liquid 123 within the drainage tube 120 to proximally move the “pooled” liquid 123 along the drainage tube 120. An air inlet tube 141 fluidly couples an inlet of the air pump 145 to the metering chamber 150, and an air outlet tube 142 fluidly couples an outlet of the air pump 145 to the drainage tube 120 at a junction point 144 adjacent the catheter 110 (i.e., the distal end of the drainage tube 120.

In some embodiments, the system 100 may include a three-way connector 146 disposed between the catheter 110 and the drainage tube 120, where the connector 146 includes a fluid flow path from the catheter 110 to the drainage tube 120. The catheter 110 may be coupled with the connector 146 at a first or distal port 146A and the distal end of the drainage tube 120 may be coupled with a second or proximal port 146B. The air outlet tube 142 may be coupled with a third or side port 146C of the connector 146 so that the catheter 110, the drainage tube 120 and the air outlet tube 142 are in fluid communication with each other. As such, the junction point 144 may be integral to the connector 146.

During air pump operation, the air pump 145 pulls air from the metering chamber 150 and delivers the air to the drainage tube 120 at the junction point 144 to define an air pressure difference across the “pooled” liquid 123 disposed in the dependent loop 122, where the pressure difference moves the “pooled” liquid 123 proximally up the positive incline 125, along the drainage tube 120, and into the metering chamber 150, i.e., the activation of the air pump 145 induces flow of the liquid 123 along the drainage tube 120.

The metering chamber 150 includes a vacuum compartment 151 and a measuring compartment 152 located below the vacuum compartment 151. A drain port 154 fluidly couples the vacuum compartment 151 to the measuring compartment 152. The drain port 154 includes a plunger valve 155 in line therewith such that the liquid 123 may passively flow through the drain port 155 from the vacuum compartment 151 to the measuring compartment 15 when the plunger valve is in a normally open state. During activation of the air pump 145, a vacuum may be generated within the vacuum compartment 151 and as a result of the vacuum, the plunger valve 155 may transition from the normally open state to a closed state to prevent fluid flow from the measuring compartment 152 to the vacuum compartment 151 via the drain port 154.

In use, in the event of pooling liquid 123 within the drainage tube 120, the air pump 145 may be activated. When the air pump 145 is activated, air is pulled from the vacuum compartment 151 and delivered to the drainage tube 120 at the junction point 144, i.e., upstream of the “pooled” liquid 123. The plunger valve 155 closes to prevent reverse fluid flow through the drain port 154. The “pooled” liquid 123 is moved into the vacuum compartment 151 accumulating therein. Thereafter, the air pump 145 is deactivated which removes the vacuum from the vacuum compartment 151. As a result, the plunger valve 151 transitions to the open state and the accumulated liquid 123 within the vacuum compartment 151 drains into the measuring compartment 152 via the drain port 154.

The metering chamber 150 includes volumetric graduation marks 157 so that a clinician may ascertain a volume of the liquid 123 contained within the measuring compartment 152. Similarly, the collection container 160 may include volumetric graduation marks 167 so that a clinician may ascertain a volume of the liquid 123 contained within the collection container 160.

The transfer port 163 extends between the measuring compartment 152 and the collection container 160. The transfer port 163 is vertically located so as to be above a defined maximum liquid level of the measuring compartment 152. In use, when the liquid 123 within the measuring compartment 152 exceeds the maximum liquid level, the liquid 123 may overflow through the transfer port 163 into the collection container 160. Further in use, the clinician may tip or rotate the metering chamber 150 to pour (i.e., empty) any and/or all of the liquid 123 contained within the measuring compartment 152 into the collection container 160 through the transfer port 163.

The metering chamber 150 includes a sample port 170 configured to facilitate obtaining a sample of the liquid 123 contained within the measuring compartment 152. The collection container 160 includes a drain tube 168 configured to facilitate selective emptying of liquid 123 from the collection container 160.

In an embodiment, the system 100 may include a safety (or isolation) valve 143 disposed between the catheter 110 and the drainage tube 120. More specifically, the safety valve 143 may be disposed between the catheter 110 and the junction point 144. The safety valve 143 may at least partially isolate the catheter 110 from the drainage tube 120. In other words, the safety valve 143, when closed, may prevent an internal pressure of the drainage tube 120 from affecting a pressure within the catheter 110. Similarly, the safety valve 143, when closed, may prevent liquid 123 within the drainage tube 120 from flowing into the catheter 110. The safety valve 143 can transition between a closed position and an open position. In the illustrated embodiment, the safety valve 143 is a check valve configured to prevent fluid flow toward the catheter 110. As such, the safety valve 143 may prevent a positive pressure, as may be present within the drainage tube 120, from reaching the patient. In some embodiments, the safety valve 143 may be integral to the connector 146. In some instances, the air pump 145, when activated, may cause a positive pressure within the drainage tube 120. As such, activating the air pump 145 may transition the safety valve 145 from the open state to the closed state.

In other embodiments, the safety valve 143 may be a manually operated valve, such as a tubing clamp or a pinch valve. In still other embodiments, the safety valve 143 may be an electro-mechanical valve, e.g., a solenoid valve or the like. In such an embodiment, the safety valve 143 may be coupled with the air pump 145 so that the safety valve 143 is actuated toward the closed position when the air pump 145 is actuated.

In some embodiments, the system 100 may include an air-port sterilizing filter 147 disposed in line with the air port 146C configured to define a sterile barrier between the drainage tube 120 and the air pump 145, i.e., the air-port sterilizing filter 147 may prevent contamination of the drainage tube 120 via the air port 146C. In some embodiments, the air-port sterilizing filter 147 may include pore size less than 0.5 microns.

In some embodiments, the system 100 include a liquid (e.g., urine) collection bag assembly 115, that includes the drainage tube 120, the metering chamber 150, and the collection container 160. Similarly, the system 100 may define air pump assembly 116, that includes the air pump 145, the air inlet tube 141 and the air outlet tube 142. The liquid collection bag assembly 115 may be configured for single use and the air pump assembly 116 may be configured for multi-use. As such, the liquid collection bag assembly 115 may be sterilized before use while the air pump assembly 116 may be used in a contaminated (i.e., non-sterile) state.

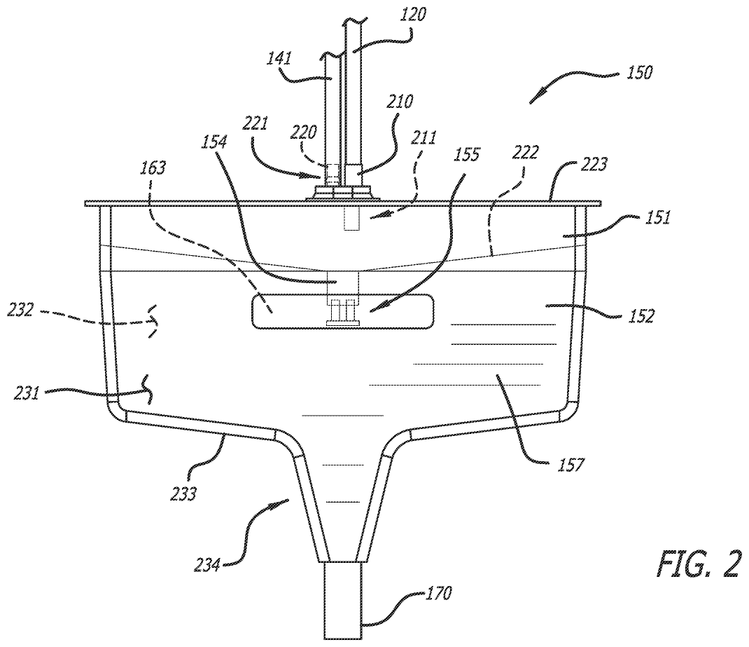

FIG. 2 illustrates a front view of the metering chamber 150. In some embodiments, the metering chamber 150 may be formed a transparent/translucent material as shown. In some embodiments, the metering chamber 150 may be formed of a rigid material, such as a rigid plastic material, for example. The metering chamber 150 includes a front wall 231 and a back wall 232, and the transfer port 163 extends through the back wall 232.

As discussed above, the metering chamber 150 includes the vacuum compartment 151 and the measuring compartment 152. A bottom wall 222 of the vacuum compartment 151 separates the vacuum compartment 151 from the measuring compartment 152 and the drain port 154 extends through the bottom wall 222. In some embodiments, the bottom wall is sloped toward the drain port 155 so that the drain port 155 my fully empty the vacuum compartment 151 of the liquid 123. The drain port 155 includes the plunger valve 155 as further described below.

A top wall 223 of the metering chamber 150 defines a cap for the vacuum compartment 151, and extending through the top wall 223 are an inlet port 210 and vacuum port 220. The inlet port 210 is coupled with the drainage tube 120. In the illustrated embodiment, the drainage tube 120 is fixedly attached to the inlet port 210. In other embodiments, the drainage tube 120 may be attachably/detachably coupled with the inlet port 210. In some embodiments, the inlet port 210 may include an internal extension 211 disposed within the vacuum compartment 151. The internal extension 211 may be configured to separate the liquid 123 from the vacuum port 220, i.e., the internal extension 211 may inhibit the liquid 123 exiting the inlet port 210 from being drawn into the vacuum port 220 during use. In the illustrated embodiment, the vacuum port 220 is attachably/detachably coupled with the air inlet tube 141. In other embodiments, the vacuum port 220 may be fixedly attached the air inlet tube 141. In the illustrated embodiment, the air inlet tube 141 may be configured for multi-use across a number of the metering chambers 150. In such embodiments, the air inlet tube 141 may be generally considered as contaminated or non-sterile. As such, in some embodiments, the vacuum port 220 may include a sterilizing filter 221 disposed in line with the vacuum port 220, where the sterilizing filter 221 is configured to prevent contamination of an interior of the metering chamber 150 via the via the vacuum port 220. In some embodiments, the sterilizing filter 221 may include a membrane defining a pore size less than 0.2 microns.

As stated above, the metering chamber 150, or more specifically the measurement compartment 152, includes the sample port 170. The sample port 170 extends through a bottom wall 233 of the measurement compartment 152, where the bottom wall 233 may be sloped toward the sample port 170. In some embodiments, the measuring compartment 152 may include a funnel portion 234 configured to accumulate a small volume of the liquid 123 toward the sample port 170.

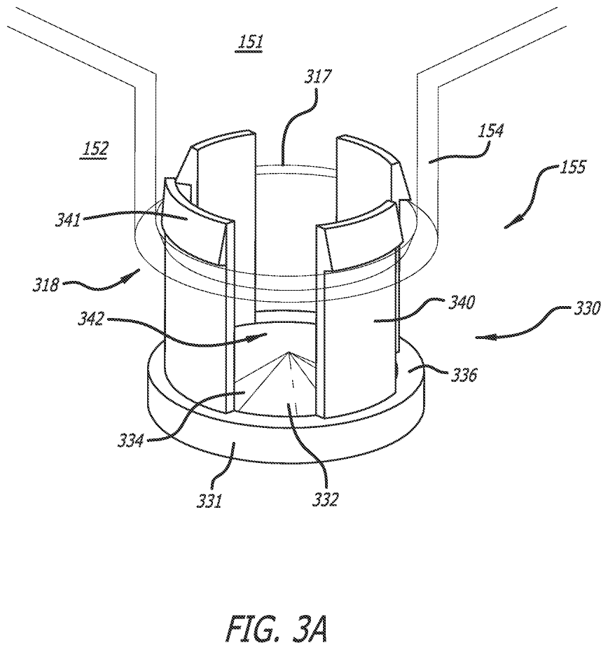

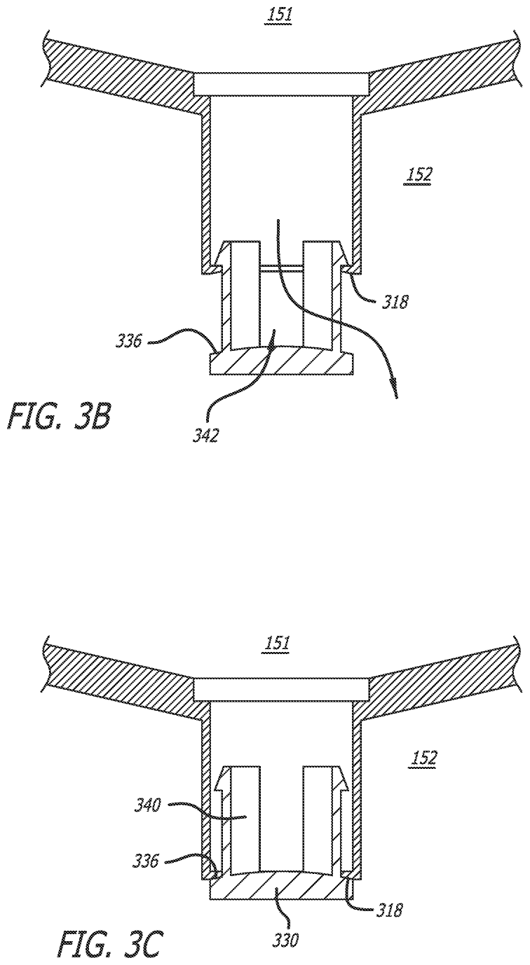

FIGS. 3A-3C illustrate various views of the plunger valve 155. FIG. 3A is a detailed perspective view of the plunger valve 155 and related components. FIG. 3B shows the plunger in a “down” or open position, and FIG. 3C shows the plunger 330 in an “up” or closed position. The plunger valve 155 includes a displaceable plunger 330 having a circular base 331. A top side 332 of the circular base 331 includes a radially outward sloped surface 334 to prevent accumulation of the liquid 123 on the top side 332 as it drains from the vacuum compartment 151.

In the illustrated embodiment, four deflectable posts 340 are coupled with and extend away from the top side 332 and each post 340 includes a hook 341 at a free end. In other embodiments, the plunger 330 may include 3, 3, 5, or more posts 340. The drain port 154 includes an inward protruding annular hook ledge 317 configured to engage the hooks 341 in a snap fit relationship. The engagement of the hooks 341 with the hook ledge 317 limits the downward displacement of the plunger 330 at the “down” position as shown in FIG. 3B. The spaces 342 between the posts 344 define a flow path for the liquid 123 as it drains from the vacuum compartment 151.

The plunger 330 further includes an annular sealing ledge 336 disposed on the top side 331 and the drain port 154 includes a corresponding annular sealing edge 318 at the bottom end of the drain port 154. The annular sealing ledge 336 and the corresponding annular sealing edge 318 are configured to the define a seal between the plunger 330 and the drain port 154 when the plunger 330 is disposed in the “up” position as show in FIG. 3C.

In use, with the plunger 330 in the “down” position, the liquid 123 drains from the vacuum compartment 151, through the drain port 154, and flows perpendicularly onto the top side 331 of the plunger 330 where it flows radially outward along the sloped surface 334 and through the spaces 342 between the posts 340 and into the measuring compartment 152. In further use, when the air pump 145 is activated, vacuum within the vacuum chamber 151 draws the plunger 330 to the “up” position, so that the annular sealing ledge 336 engages the corresponding annular sealing edge 318 to seal off the drain port 312, thereby preventing air and/or liquid 123 from flowing upward through the drain port 154.

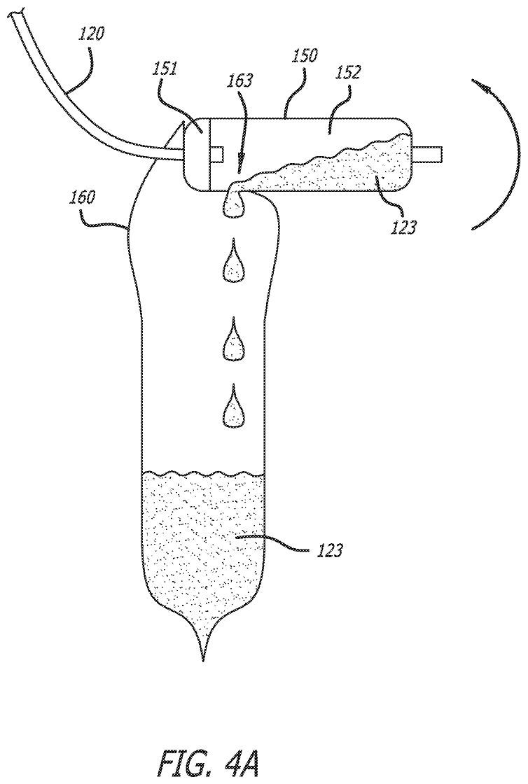

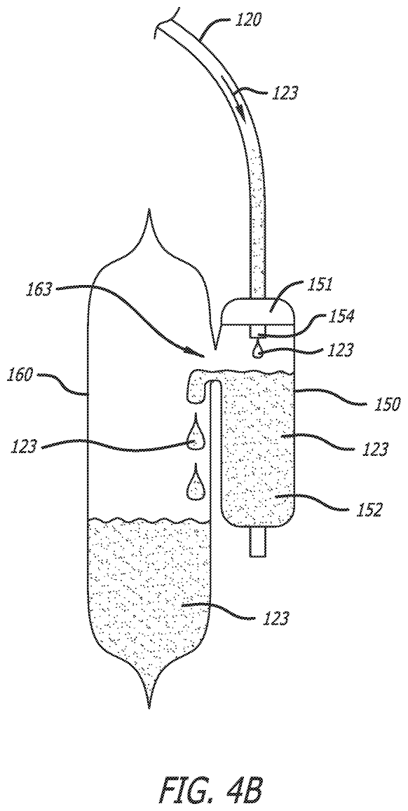

FIG. 4A illustrates the cross-sectional side view of the system 100 in a first instance of use. The system 100 is generally configured to (i) collect an amount of liquid 123 drained from a patient within the measuring compartment 152 over a defined measurement time period, such as one hour, for example, and (ii) collect a substantial entirety of the drainage liquid 123 over a defined collection period (e.g., 8 or 24 hours) within the collection container 160. At the conclusion of the measurement time period, the clinician may record a volumetric measurement of the amount of liquid 123 within the measuring compartment 152. Thereafter, the clinician may transfer the amount of liquid 123 from the measuring compartment 152 to the collection container 160. To transfer the amount of liquid 123, the clinician may rotate or tip the metering chamber 150 away from a normally vertical orientation toward a horizontal orientation as illustrated in FIG. 4A to pour the first amount of liquid 123 out of the measuring compartment 152 into the collection container 160 through the transfer port 163. The process of collecting an amount of liquid 123 in the measuring compartment 152, recording the volumetric measurement, and pouring the amount of liquid 123 into the collection container 160 may be repeated several times over a the defined collection period.

FIG. 4B illustrates a cross-sectional side view of the system 100 in a second instance of use. In some instances, a patient may excrete liquid 123 sufficient to exceed the capacity of the measurement compartment 152. In such an instance, the liquid 123 may overflow the measurement compartment 152 through the transfer port 163 and into the collection container 160 as shown.

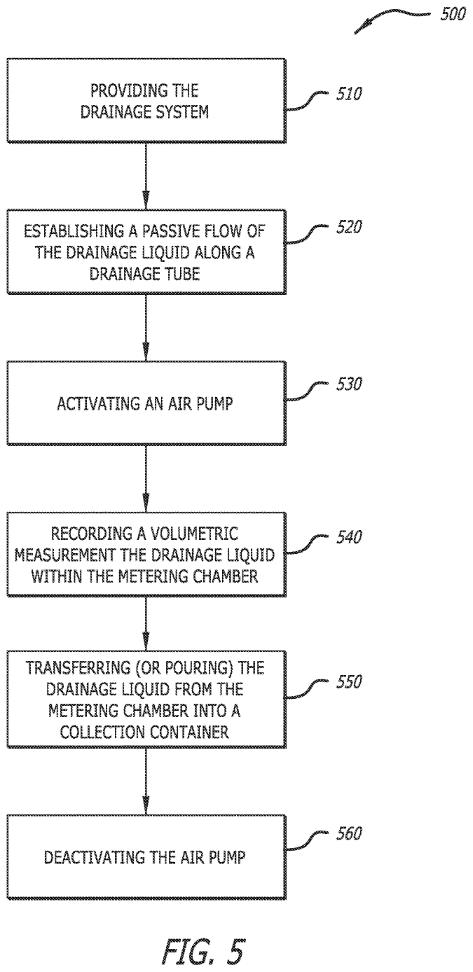

FIG. 5 illustrates a block diagram of a method of using the system 100 to drain liquid from a patient that, according to some embodiments, includes all or any subset of the following steps, actions or processes. The method 500 may include providing the drainage system (block 510) where the drainage system includes the drainage tube extending between the catheter and the metering chamber. The method further includes establishing a passive flow of the drainage liquid from the patient along the drainage tube (block 520). In some instances, the passive flow continues unimpeded throughout the drainage process. In some instances, the drainage liquid may pool within the drainage tube. As such, the method 500 may include activating an air pump (block 530) to transport the pooled drainage liquid within the drainage tube to the metering chamber. Activating the air pump defines a pressure difference across the pooled drainage liquid within the drainage tube, and activating the air pump also transitions the plunger valve disposed between a vacuum compartment and a measuring compartment of the metering chamber from an open state to closed state. The method 500 may further include recording a volumetric measurement the drainage liquid contained within the metering chamber (block 540). The method 500 may further include transferring (or pouring) the drainage liquid from the metering chamber into a collection container (block 550) where the drainage liquid flows through the transfer port from the metering chamber to the collection container.

In some embodiments, the method 500 further includes deactivating the air pump (block 560) to transition the plunger valve from the closed state to the open state to allow the liquid collected within the vacuum compartment to flow into the measuring compartment through the plunger valve in the open state.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A drainage system for draining a liquid from a patient, the drainage system comprising:
a drainage tube configured to receive a drainage liquid from the patient, a distal end of the drainage tube configured to couple with a catheter;
a metering chamber coupled with the drainage tube at the proximal end, the metering chamber comprising:
a vacuum compartment, comprising:
an inlet port coupled with the drainage tube;
a vacuum port; and
a drain port including a plunger valve in line therewith; and
measuring compartment fluidly coupled with the vacuum chamber via the drain port;
a collection container coupled with the metering chamber via a transfer port configured to enable selective transfer of the drainage liquid from the measuring compartment to the collection container;
an air pump assembly, comprising:
an air pump having an air inlet and an air outlet;
an air inlet tube coupled between the air inlet and the vacuum port: and
an air outlet tube coupled between the air outlet and an air port of the drainage tube adjacent the distal end, wherein:
upon activation of the air pump, flow of the drainage liquid is induced along the drainage tube toward the vacuum chamber, and
upon deactivation of the air pump, drainage liquid drains from the vacuum compartment into the measuring compartment through the plunger valve.

2. The system according to claim 1, wherein the plunger valve is configured to transition between:
an open state when the air pump is deactivated, and
a closed state when the air pump is deactivated.

3. The system according to claim 1, wherein the plunger valve comprises a plunger displaceable between a “down” position in accordance with the plunger valve disposed in the open state, and an “up” position in accordance with the plunger valve disposed in the closed state.

4. The system according to claim 3, wherein the plunger comprises a plurality of deflectable posts coupled with a circular base, the posts extending vertically away from the circular base.

5. The system according to claim 4, wherein the posts are coupled with the drainage port via a snap fit coupling, the snap fit coupling configured to limit downward displacement of the plunger, defining the “down” position.

6. The system according to claim 4, wherein the circular base comprises an annular sealing ledge extending along a circumference of the circular base, the annular sealing ledge configured to form a seal with a corresponding annular sealing edge of the drain port when the plunger is disposed in the “up” position, the seal defining the closed state of the plunger valve.

7. The system according to claim 3, wherein:
activation of the air pump defines a vacuum within the vacuum compartment, the vacuum exerting an upward force on the plunger to displace the plunger to the “up” position, and
deactivation of the air pump relieves the vacuum within the vacuum compartment, allowing the plunger to self-displace to the “down” position.

8. The system according to claim 1, wherein the metering chamber is coupled to a front side of the collection container.

9. The system according to claim 1, wherein a front wall of the measuring compartment includes volumetric graduation marks configured to indicate a volume of the drainage liquid contained within the measuring compartment.

10. The system according to claim 1, wherein the transfer port is located above a defined maximum liquid level of the measuring compartment.

11. The system according to claim 1, wherein a front wall of the collection container includes volumetric graduation marks configured to indicate a volume of the drainage liquid contained within the collection container.

12. The system according to claim 1, wherein the transfer port is located above a defined maximum liquid level of the collection container.

13. The system according to claim 1, wherein a bottom wall of the vacuum compartment is sloped toward the drain port to facilitate complete drainage of the drainage liquid from the vacuum compartment.

14. The system according to claim 1, wherein:
a bottom wall of the measuring compartment includes a sample port, and
the bottom wall of the measuring compartment is sloped toward the sample port to facilitate sampling of the drainage liquid within the measuring compartment.

15. The system according to claim 1, further comprising an isolation valve disposed in line with the drainage tube adjacent the distal end, the isolation valve configured to prevent fluid flow toward the catheter.

16. The system according to claim 15, wherein the isolation valve is configured to transition between a closed state and open state in accordance with activation and deactivation of the air pump, respectively.

17. The system according to claim 15, wherein the isolation valve is a check valve configured to allow proximal fluid flow through the isolation valve and prevent distal fluid flow through the isolation valve.

18. The system according to claim 15, further comprising a connector disposed at the distal end of the drainage tube, wherein the air port and the isolation valve are integral to the connector.

19. The system according to claim 1, further comprising a vacuum-port sterilizing filter disposed in line with the vacuum port, the vacuum-port sterilizing filter configured to inhibit contamination of the metering chamber via the vacuum port.

20. The system according to claim 1, further comprising an air-port sterilizing filter disposed in line with the air port, the air-port sterilizing filter configured to inhibit contamination of the drainage tube via the air port.

21-38. (canceled)
